(19) United States
(12) Patent Application Publication
(10) Pub. No.: US 2008/0004348 A1
(43) Pub. Date: Jan. 3, 2008

(54) Naphthalene compounds

(75) Inventors: Said Yous, Loos (FR); Basile Peres, Foix (FR); Ahmed Sabaouni, Armentieres (FR); Pascal Berthelot, Haubourdin (FR); Michael Spedding, Le Vesinet (FR); Philippe Delagrange, Issy Les Moulineaux (FR); Daniel-Henri Caignard, Boisemont (FR); Matthieu Desroses, Lille (FR); Jean-Albert Boutin, Suresnes (FR); Valerie Audinot, Poissy (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE CEDEX (FR)

(21) Appl. No.: 11/823,130

(22) Filed: Jun. 27, 2007

(30) Foreign Application Priority Data
Jun. 30, 2006 (FR) 0605916

(51) Int. Cl. A61K 31/16; C07C 235/32

(52) U.S. Cl. 514/630; 564/219

(57) ABSTRACT

A compound selected from those of formula (I):

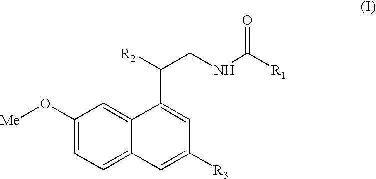

wherein:
R₁ represents a group R₄ or NHR₄, wherein R₄ is as defined in the description;
R₂ represents a substituted linear or branched (C₁-C₆)alkyl group;
R₃ represents a hydrogen or halogen atom or a linear or branched (C₁-C₆)alkyl or linear or branched (C₂-C₆)alkenyl group.

Medicinal products containing the same which are useful in the treatment of disorders of the melatoninergic system.

The present invention relates to new naphthalene compounds, to a process for their preparation and to pharmaceutical compositions containing them.

The compounds of the present invention are new and have very valuable pharmacological characteristics relating to melatoninergic receptors.

Numerous studies in the last ten years have demonstrated the key role of melatonin (N-acetyl-5-methoxytryptamine) in many physiopathological phenomena and in the control of circadian rhythms. Its half-life is quite short, however, owing to the fact that it is rapidly metabolised. Great interest therefore lies in the possibility of providing the clinician with melatonin analogues that are metabolically more stable, have an agonist or antagonist character and may be expected to have a therapeutic effect that is superior to that of the hormone itself.

In addition to their beneficial action on circadian rhythm disorders (J. Neurosurg. 1985, 63, pp. 321-341) and sleep disorders (Psychopharmacology, 1990, 100, pp. 222-226), ligands of the melatoninergic system have valuable pharmacological properties in respect of the central nervous system, especially anxiolytic and antipsychotic properties (Neuropharmacology of Pineal Secretions, 1990, 8 (3-4), pp. 264-272) and analgesic properties (Pharmacopsychiat., 1987, 20, pp. 222-223) as well as for the treatment of Parkinson's disease (J. Neurosurg. 1985, 63, pp. 321-341) and Alzheimer's disease (Brain Research, 1990, 528, pp. 170-174). Those compounds have also demonstrated activity in respect of certain cancers (Melatonin—Clinical Perspectives, Oxford University Press, 1988, pp. 164-165), ovulation (Science 1987, 227, pp. 714-720), diabetes (Clinical Endocrinology, 1986, 24, pp. 359-364), and in the treatment of obesity (International Journal of Eating Disorders, 1996, 20 (4), pp. 443-446).

Those various effects are exerted via the intermediary of specific melatonin receptors. Molecular biology studies have demonstrated the existence of a number of receptor sub-types that are capable of binding that hormone (Trends Pharmacol. Sci., 1995, 16, p. 50; WO 97.04094). For various species, including mammals, it has been possible for some of those receptors to be located and characterised. In order to be able to understand the physiological functions of those receptors better, it is of great advantage to have available selective ligands. Moreover, such compounds, by interacting selectively with one or other of those receptors, may be excellent medicaments for the clinician in the treatment of pathologies associated with the melatoninergic system, some of which have been mentioned above.

Besides the fact that they are new, the compounds of the present invention exhibit a very strong affinity for melatonin receptors and/or selectivity for one or other of the melatoninergic binding sites.

They moreover have a strong affinity for the 5-HT_{2C} receptor, which has the effect of reinforcing the properties observed in the case of melatoninergic receptors, especially in the field of depression.

More specifically, the present invention relates to compounds of formula (I):

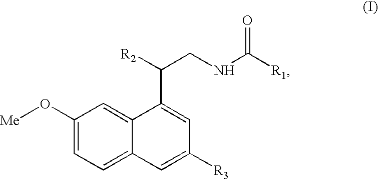

wherein:
R₁ represents a group R₄ or NHR₄, wherein R4 represents a linear or branched (C₁-C₆)-alkyl group, a linear or branched (C₁-C₆)alkenyl group, a linear or branched (C₁-C₆)-haloalkyl group, a linear or branched (C₁-C₆)polyhaloalkyl group, a (C₃-C₈)cycloalkyl group, a (C₃-C8)cycloalkyl-(C₁-C₆)alkyl group in which the alkyl moiety may be linear or branched, an aryl group, an aryl-(C₁-C₆)alkyl group in which the alkyl moiety may be linear or branched, a heteroaryl group or a heteroaryl-(C₁-C₆)alkyl group in which the alkyl moiety may be linear or branched,
R₂ represents a linear or branched (C₁-C₆)alkyl group substituted by a linear or branched (C₁-C₆)alkoxy group, OH, OSO₂Me, N₃, NRR′, NHCOR″ or by NHSO₂R″, wherein R and R′, which may be the same or different, each represent a hydrogen atom or a linear or branched (C₁-C₆)alkyl group, a (C₃-C₈)cycloalkyl group, an aryl group or an aryl(C₁-C₆)alkyl group in which the alkyl moiety may be linear or branched, or R and R′ together with the nitrogen atom carrying them form a 5- or 6-membered ring which may contain another hetero atom selected from nitrogen, oxygen and sulphur, and R″ represents a linear or branched (C₁-C₆)alkyl group, a (C₃-C₈)cycloalkyl group, an aryl group or an aryl(C₁-C₆)alkyl group in which the alkyl moiety may be linear or branched,
R₃ represents a hydrogen or halogen atom or a linear or branched (C₁-C₆)alkyl or linear or branched (C₁-C₆)alkenyl group,

it being understood that:
when R₁ represents a methyl group and R₂ represents a hydroxymethyl group, then R₃ cannot represent a hydrogen atom,
“aryl” means a phenyl, naphthyl or biphenyl group,
“heteroaryl” means any aromatic mono- or bi-cyclic group containing from 1 to 3 hetero atoms selected from oxygen, sulphur and nitrogen,

it being possible for the aryl and heteroaryl groups so defined to be substituted by from 1 to 3 groups selected from linear or branched (C₁-C₆)alkyl, linear or branched (C₁-C₆)alkoxy, hydroxy, carboxy, formyl, nitro, cyano, linear or branched (C₁-C₆) haloalkyl, linear or branched (C₁-C₆)polyhaloalkyl, alkyloxycarbonyl and halogen atoms,

to their enantiomers and diastereoisomers, and also to addition salts thereof with a pharmaceutically acceptable acid or base.

Among the pharmaceutically acceptable acids there may be mentioned by way of non-limiting example hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned by way of non-limiting example sodium hydroxide, potassium hydroxide, triethylamine, tert-butylamine etc.

Preferred compounds of the invention are compounds of formula (I) wherein R₁ represents a linear or branched (C₁-C₆)alkyl group such as, for example, a methyl or ethyl group.

R₂ advantageously represents an alkyl group substituted by an OH or alkoxy group.

R₃ advantageously represents a hydrogen atom.

The invention even more specifically relates to the compounds which are N-[3-methoxy-2-(7-methoxy-1-naphthyl)propyl]acetamide, N-[4-hydroxy-2-(7-methoxy-1-naphthyl)butyl] propanamide and N-[4-hydroxy-2-(7-methoxy-1-naphthyl)butyl]acetamide.

The enantiomers and diastereoisomers and the addition salts of preferred compounds of the invention with a pharmaceutically acceptable acid or base form an integral part of the invention.

The invention relates also to a process for the preparation of compounds of formula (I), which process is characterised in that there is used as starting material the compound of formula (II):

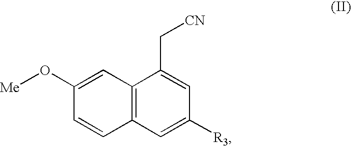

wherein R₃ is as defined for formula (I), which is subjected to the action of dimethyl carbonate in a basic medium to yield the compound of formula (III):

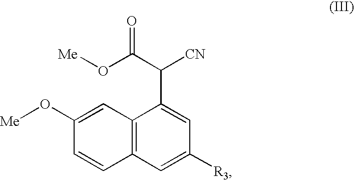

wherein R₃ is as defined hereinbefore, with which optionally there is condensed a compound of formula Hal-(CH₂)ₙ—COOMe, wherein Hal represents a halogen atom and n is from 1 to 6, to yield the compound of formula (IV):

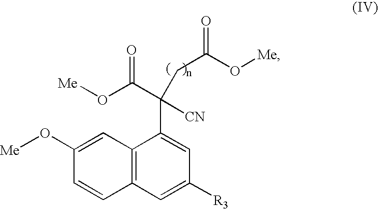

wherein R₃ and n are as defined hereinbefore,

which is subjected to the action of lithium bromide to yield the compound of formula (V):

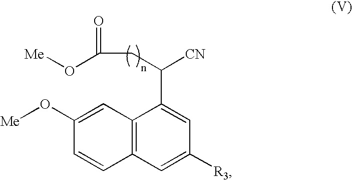

wherein R₃ and n are as defined hereinbefore, the totality of compounds of formulae (III) and (V) forming the compound of formula (VI):

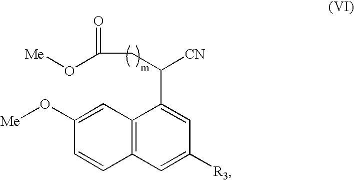

wherein R₃ is as defined hereinbefore and m is 0, 1, 2, 3, 4, 5 or 6,

which is subjected to reduction in the presence of a hydride to yield the compound of formula (VII):

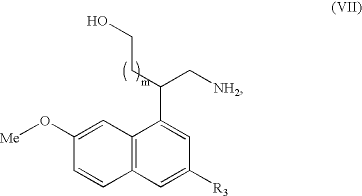

wherein R₃ and m are as defined hereinbefore,

with which there is condensed a compound of formula R₁C(O)Cl to yield the compound of formula (I/a), a particular case of the compounds of formula (I)

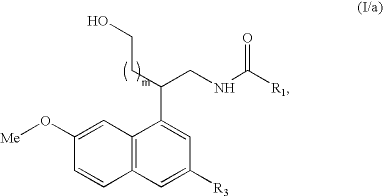

wherein R₃, m and R₁ are as defined hereinbefore, which optionally is
either subjected to the action of an alkyl halide in a basic medium to yield the compound of formula (I/b), a particular case of the compounds of formula (I):

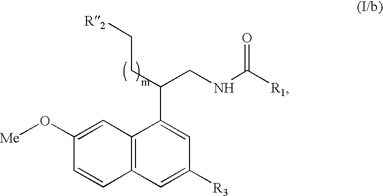

wherein R₃, m and R₁ are as defined hereinbefore and R″₂ represents a linear or branched (C₁-C₆)alkoxy group,
or condensed with mesylate chloride in a basic medium to yield the compound of formula (I/c), a particular case of the compounds of formula (I):

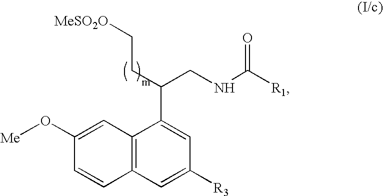

wherein R₃, m and R₁ are as defined hereinbefore, with which optionally there is condensed:
either an amine of formula HNRR′, wherein R and R′ are as defined for formula (I), to yield the compound of formula (I/d), a particular case of the compounds of formula (I)

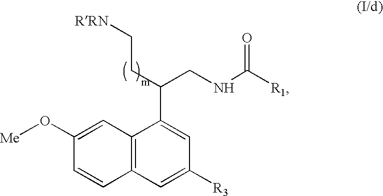

wherein R₃, m, R, R′ and R₁ are as defined hereinbefore,
or an azide to yield the compound of formula (I/e), a particular case of the compounds of formula (I)

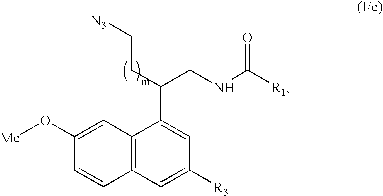

wherein R₃, m and R₁ are as defined hereinbefore,

which optionally is subjected to reduction in the presence of palladium-on-carbon, optionally followed by mono- or bis-condensation with a compound of formula R-Hal, wherein R is as defined for formula (I), to yield a compound of formula (I/d) as defined hereinbefore wherein R and R′ do not form a cyclic group together with the nitrogen atom carrying them,

which compound of formula (I/d), when R and R′ simultaneously represent a hydrogen atom, is optionally subjected to the action of a compound of formula R″C(O)Cl or

R″SO₂Cl, wherein R″ is as defined hereinbefore, to yield the compound of formula (I/f, a particular case of the compounds of formula (I):

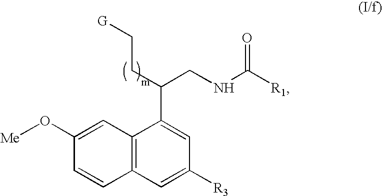

wherein R₃, m and R₁ are as defined hereinbefore and G represents a group NHCOR″ or NHSO₂R″, wherein R″ is as defined for formula (I),

the compounds of formulae (I/a) to (I/f) forming the totality of the compounds of formula (I), which may be purified according to a conventional separation technique, which are converted, if desired, into their addition salts with a pharmaceutically acceptable acid or base, and which are separated, where appropriate, into their isomers according to a conventional separation technique.

The compounds of formula (II) are either commercially available or can be obtained by the person skilled in the art using conventional chemical reactions described in the literature.

Pharmacological study of the compounds of the invention has shown them to be a toxic, to have strong selective affinity for melatonin receptors and to have significant activities in respect of the central nervous system; and, in particular, therapeutic properties in respect of sleep disorders, antidepressive, anxiolytic, antipsychotic and analgesic properties and properties in respect of microcirculation have been found, enabling it to be established that the compounds of the invention are useful in the treatment of stress, sleep disorders, anxiety, seasonal affective disorder or major depression, cardiovascular pathologies, pathologies of the digestive system, insomnia and fatigue due to jetlag, schizophrenia, panic attacks, melancholia, appetite disorders, obesity, insomnia, psychotic disorders, epilepsy, diabetes, Parkinson's disease, senile dementia, various disorders associated with normal or pathological aging, migraine, memory loss and Alzheimer's disease, and in cerebral circulation disorders. In another field of activity, it appears that, in treatment, the compounds of the invention can be used in sexual dysfunctions, that they have ovulation-inhibiting and immunomodulating properties and that they may potentially be used in the treatment of cancers.

The compounds will preferably be used in the treatment of major depression, seasonal affective disorder, sleep disorders, cardiovascular pathologies, pathologies of the digestive system, insomnia and fatigue due to jetlag, appetite disorders and obesity.

For example, the compounds will be used in the treatment of major depression, seasonal affective disorder and sleep disorders.

The present invention relates also to pharmaceutical compositions comprising at least one compound of formula (I) on its own or in combination with one or more pharmaceutically acceptable excipients.

Among the pharmaceutical compositions according to the invention there may be mentioned more especially those that are suitable for oral, parenteral, nasal, per- or trans-cutaneous, rectal, perlingual, ocular or respiratory administration and especially tablets or dragées, sublingual tablets, sachets, paquets, capsules, glossettes, lozenges, suppositories, creams, ointments, dermal gels, and drinkable or injectable ampoules.

The dosage varies according to the sex, age and weight of the patient, the route of administration, the nature of the therapeutic indication or any associated treatments and ranges from 0.01 mg to 1 g per 24 hours in one or more administrations.

The following Examples illustrate the invention but do not limit it in any way.

### Example 1

### N-[3-Hydroxy-2-(7-methoxy-1-naphthyl)propyl]cyclopropane-carboxamide

### Step A: Methyl cyano(7-methoxy-1-naphthyl)acetate

(7-Methoxy-naphth-1-yl)acetonitrile (20 g) is dissolved in 150 ml of anhydrous tetrahydrofuran. Sodium hydride (202.8 mmol) is added at ambient temperature, and the mixture is refluxed for 30 minutes. Dimethyl carbonate (12 ml) is added with caution, and the reaction mixture is refluxed for 30 minutes. The mixture is poured into ice-cold water, and the aqueous phase is acidified with 21 ml of 37% hydrochloric acid solution and then extracted twice with 100 ml of ether. The organic phase is washed with water, dried, decoloured and evaporated. The oil obtained is precipitated from ether, and the precipitate formed is filtered off under suction and then recrystallised to yield the title compound in the form of a white solid.

Melting Point: 80-82° C.

### Step B: 3-Amino-2-(7-methoxy-1-naphthyl)-1-propanol hydrochloride

Aluminium chloride (80 mmol), dissolved in 200 ml of anhydrous ether, is added to a suspension of lithium aluminium hydride at 0° C. in 300 ml of anhydrous ether. After stirring for 10 minutes, the compound obtained in Step A (20 mmol), dissolved in 200 ml of anhydrous ether, is added. After 30 minutes, the mixture is hydrolysed, with caution and in the cold state, using sodium hydroxide solution (10 g; 40 ml). The precipitate formed is then filtered off and washed with copious amounts of ether. The residue obtained after evaporation is taken up in water and the aqueous phase is extracted with dichloromethane. The organic phase is then washed with water, dried and decoloured, and is then treated with gaseous hydrogen chloride and evaporated. The oil obtained is precipitated from ethyl acetate, and the precipitate formed is filtered off under suction and then recrystallised to yield the title compound in the form of a white solid.

Melting Point: 164-166° C.

### Step C: N-[3-Hydroxy-2-(7-methoxy-1-naphthyl)propyl]cyclopropane-carboxamide

The compound obtained in Step B (3.73 mmol) is dissolved in 100 ml of a mixture of water/ethyl acetate (50/50). Potassium carbonate (11.2 mmol) is added and the reaction mixture is cooled to 0° C. using an ice bath. Cyclopropanoyl chloride (4.4 mmol) is added dropwise and the mixture is stirred for 15 minutes in the cold state. When the reaction is complete, the organic phase is washed with hydrochloric acid solution (1M), washed with water, dried and evaporated under reduced pressure. The solid obtained is recrystallised from toluene to yield the title compound in the form of a white solid.

Melting Point: 153-154° C.

Elemental Microanalysis:

| % | C | H | N |
|---|---|---|---|
| Calculated: | 72.22 | 7.07 | 4.68 |
| Found: | 72.38 | 7.28 | 4.53 |

### Example 2

### N-[3-Hydroxy-2-(7-methoxy-1-naphthyl)propyl]acrylamide

The procedure is as in Step C of Example 1, replacing cyclopropanoyl chloride by acryloyl chloride.

Melting Point: 150-152° C.

Elemental Microanalysis:

| % | C | H | N |
|---|---|---|---|
| Calculated: | 71.56 | 6.71 | 4.91 |
| Found: | 71.37 | 6.81 | 4.82 |

### Example 3

### N-[3-Hydroxy-2-(7-methoxy-1-naphthyl)propyl]-3-butenamide

Vinylacetic acid (5 mmol) is dissolved in 40 ml of dichloromethane and the solution is cooled to 0° C. EDCI (6 mmol) is added in small portions and the mixture is stirred at 0° C. for 30 minutes. The compound obtained in Step B of Example 1, in the form of the base and dissolved in 20 ml of dichloromethane, is added to the mixture. After stirring for 30 minutes at 0° C., the reaction mixture is poured into water. The organic phase is washed with hydrochloric acid solution (1M) and then with sodium hydrogen carbonate solution (1M) and with water. The organic phase is then dried and evaporated. The oil obtained is precipitated from ether, and the solid obtained is filtered off under suction and then recrystallised from toluene to yield the title compound in the form of a white solid.

Melting Point: 86-88° C.

### Example 4

### 2,2,2-Trifluoro-N-[3-hydroxy-2-(7-methoxy-1-naphthyl)propyl]-acetamide

The compound obtained in Step B of Example 1 (5.6 mmol) is dissolved in 40 ml of tetrahydrofuran in the presence of triethylamine (11.2 mmol), and trifluoroacetic anhydride (5 mmol) is added. The mixture is stirred at ambient temperature for 10 minutes, concentrated under reduced pressure and then poured into water. The aqueous phase is extracted twice with 60 ml of ether, and the organic phase is washed with hydrochloric acid solution (1M) and is then washed with water, dried and evaporated. The oil obtained is precipitated from a mixture of ether/petroleum ether (50/50), and the precipitate formed is filtered off under suction and then recrystallised from toluene to yield the title compound in the form of a white solid.

Melting Point: 138-140° C.

### Example 5

### 4-Chloro-N-[3-hydroxy-2-(7-methoxy-1-naphthyl)propyl]butanamide

The procedure is as in Step C of Example 1, replacing the cyclopropanoyl chloride by 4-chlorobutanoyl chloride.

Whitish Oil

### Example 6

### N-[3-Methoxy-2-(7-methoxy-1-naphthyl)propyl]acetamide

### Step A: 3-Methoxy-2-(7-methoxy-1-naphthyl)-1-propanamine hydrochloride

The title compound is obtained by alkylation of the compound obtained in Step B of Example 1.

### Step B: N-[3-Methoxy-2-(7-methoxy-1-naphthyl)propyl]acetamide

The procedure is as in Step C of Example 1, starting from the compound obtained in Step A and replacing the cyclopropanoyl chloride by acetyl chloride.

Melting Point: 82-84° C.

Elemental Microanalysis

| % | C | H | N |
|---|---|---|---|
| Calculated: | 71.05 | 7.36 | 4.87 |
| Found: | 70.77 | 7.57 | 4.78 |

### Example 7

### N-[4-Hydroxy-2-(7-methoxy-1-naphthyl)butyl]acetamide

### Step A: Dimethyl 2-cyano-2-(7-methoxy-1-naphthyl)succinate

The compound obtained in Step A of Example 1 (19.6 mmol) is dissolved in 80 ml of acetone in the presence of tetrabutylammonium bromide (200 mg) and potassium carbonate (58.8 mmol). The mixture is refluxed for 20 minutes and methyl bromoacetate (23.5 mmol) is added dropwise. The mixture is held at reflux for 10 minutes and filtered when the reaction is complete. The potassium carbonate is washed with acetone and the filtrate is evaporated. The solid obtained is filtered off from ether under suction and is then recrystallised from a mixture of toluene/cyclohexane (3/2) to yield the title compound in the form of a beige solid.

Melting Point: 119-121° C.

### Step B: Methyl 3-cyano-3-(7-methoxy-1-naphthyl)propanoate

The compound obtained in Step A (16 mmol) is dissolved in 15 ml of dimethylformamide, and then lithium bromide (16 mmol) and water (16 mmol) are added to the solution. The reaction mixture is then refluxed for 16 hours and poured into 30 ml of water when the reaction is complete. The precipitate formed is dried and then recrystallised from a mixture of toluene/cyclohexane (1/3) to yield the title compound in the form of a beige solid.

Melting Point: 113-114° C.

### Step C: Methyl 4-(acetylamino)-3-(7-methoxy-1-naphthyl)butanoate

The compound obtained in Step B (18.5 mmol) is dissolved in 200 ml of acetic anhydride, and Raney nickel (2 g) is added to the solution. The reaction mixture is placed under a pressure of 30 bar of hydrogen and heated at 60° C. for 3 hours, and is then filtered and evaporated to dryness. The residue obtained is taken up in 100 ml of water and the aqueous phase is extracted twice with 100 ml of ether. The organic phase is washed with sodium hydrogen carbonate solution (1M), washed with water, dried and evaporated. The oil obtained is precipitated from ether, and the precipitate formed is filtered off under suction and then recrystallised from diisopropyl ether to yield the title compound in the form of a white solid.

Melting Point: 94-95° C.

### Step D: N-[4-Hydroxy-2-(7-methoxy-1-naphthyl)butyl]acetamide

The compound obtained in Step C (6.3 mmol) is dissolved in 200 ml of anhydrous ether, and lithium aluminium hydride (9.45 mmol) is added in small portions. The mixture is stirred at ambient temperature for 6 hours and is neutralised using 2 ml of water. The ether phase is washed with water, dried and evaporated. The oil obtained is purified on silica gel using a mixture of acetone/ethyl acetate (40/60) as eluant to yield the title compound in the form of a white solid.

Melting Point: 143-145° C.

### Example 8

### 3-(Acetylamino)-2-(7-methoxy-1-naphthyl)propyl methanesulphonate

### Step A: 3-Amino-2-(7-methoxy-1-naphthyl)propyl methanesulphonate hydrochloride

The title compound is obtained by mesylation of the compound obtained in Step B of Example 1.

### Step B: 3-(Acetylamino)-2-(7-methoxy-1-naphthyl)propyl methanesulphonate

The procedure is as in Step C of Example 1, starting from the compound obtained in Step A and replacing the cyclopropanoyl chloride by acetyl chloride. The title compound is obtained in the form of a white solid.

Melting Point: 104-106° C.

### Example 9

### 2-(7-Methoxy-1-naphthyl)-3-(propionylamino)propyl methanesulphonate

The procedure is as in Example 8, replacing the acetyl chloride in Step B by propanoyl chloride. The title compound is obtained in the form of a white solid.

Melting Point: 118-120° C.

### Example 10

### N-[2-(7-Methoxy-1-naphthyl)-3-(4-morpholinyl)propyl]propanamide hydrochloride

The compound obtained in Example 8 (4.26 mmol) and morpholine (42.3 mmol) are dissolved in 40 ml of anhydrous tetrahydrofuran, and the reaction mixture is refluxed under a current of argon for 24 hours. When the reaction is complete, the mixture is concentrated in vacuo and then poured into water. The aqueous phase is extracted twice with 50 ml of ether, and the organic phase is washed with water and then washed with hydrochloric acid solution (1M). The aqueous phase is then rendered alkaline with 15 % sodium hydroxide solution and is then extracted twice with 50 ml of ether. The organic phase is washed with water, dried, decoloured and then treated with ether saturated with HCl. The precipitate formed is filtered off under suction and is then recrystallised from acetonitrile to yield the title compound in the form of a white solid.

Melting point: 125-126° C.

Elemental Microanalysis:

| % | C | H | N |
|---|---|---|---|
| Calculated: | 63.40 | 7.18 | 7.39 |
| Found: | 63.38 | 7.22 | 7.37 |

### Example 11

### N-[3-azido-2-(7-methoxy-1-naphthyl)propyl]acetamide

Sodium azide (25.6 mmol) is suspended in 10 ml of dimethylformamide; tetrabutyl-ammonium bromide (a spatula tip) is added and the mixture is heated to 70° C. The compound obtained in Example 8 (8.53 mmol), dissolved in 20 ml of dimethylformamide, is then added and the mixture is stirred at 70° C. for two hours. When the reaction is complete, 40 ml of water are added and the aqueous phase is extracted three times with 60 ml of ether. The organic phase is then washed with hydrochloric acid solution (2M) and then with water. After having been dried, the organic phase is evaporated to yield the title compound in the form of an orange-coloured oil.

Orange-coloured oil

### Example 12

### N-[3-Azido-2-(7-methoxy-1-naphthyl)propyl]propanamide

The procedure is as in Example 11, starting from the compound obtained in Example 9.

Whitish oil

### Example 13

### N-[3-Amino-2-(7-methoxy-1-naphthyl)propyl]acetamide hydrochloride

The compound obtained in Example 11 (6.48 mmol) is dissolved in 50 ml of methanol, and palladium-on-carbon (200 mg) is added to the solution. The mixture is then placed under hydrogen at atmospheric pressure and stirred at ambient temperature for 2 hours. When the reaction is complete, the catalyst is filtered off and the methanol is evaporated off. The residue obtained is taken up in ether and the resulting insoluble material is filtered off. The filtrate is then treated with ether saturated with HCl and the hydrochloride formed is filtered off under suction and then recrystallised from a mixture of acetonitrile/methanol (8/2) to yield the title compound in the form of a white solid.

Melting Point: 230-231 ° C.

### Example 14

### N-[3-Amino-2-(7-methoxy-1-naphthyl)propyl]propanamide hydrochloride

The procedure is as in Example 13, starting from the compound obtained in Example 12.

Melting Point: 198-200° C.

### Example 15

### N-[3-(Acetylamino)-2-(7-methoxy-1-naphthyl)propyl]acetamide

The procedure is as in Step C of Example 1, starting from the compound obtained in Example 13 and replacing the cyclopropanoyl chloride by acetyl chloride. The title compound is recrystallised from acetonitrile and obtained in the form of a white solid.

Melting Point: 199-200° C.

Elemental Microanalysis:

| % | C | H | N |
|---|---|---|---|
| Calculated: | 68.77 | 7.05 | 8.91 |
| Found: | 68.67 | 7.15 | 8.89 |

### Example 16

### N-[3-(Acetylamino)-2-(7-methoxy-1-naphthyl)propyl]propanamide

The procedure is as in Example 15, starting from the compound obtained in Example 13 and replacing the acetyl chloride by propanoyl chloride. The title compound is recrystallised from acetonitrile and obtained in the form of a white solid.

Melting Point: 148-150° C.

Elemental Microanalysis:

| % | C | H | N |
|---|---|---|---|
| Calculated: | 69.49 | 7.37 | 8.53 |
| Found: | 69.74 | 7.46 | 8.42 |

### Example 17

### N-[3-(Acetylamino)-2-(7-methoxy-1-naphthyl)propyl]butanamide

The procedure is as in Example 15, starting from the compound obtained in Example 13 and replacing the acetyl chloride by butanoyl chloride. The title compound is recrystallised from acetonitrile and obtained in the form of a white solid.

Melting Point: 150-152° C.

Elemental Microanalysis:

| % | C | H | N |
|---|---|---|---|
| Calculated: | 70.15 | 7.65 | 8.18 |
| Found: | 70.22 | 7.33 | 8.25 |

### Example 18

### N-[3-(Acetylamino)-2-(7-methoxy-1-naphthyl)propyl]cyclopropane-carboxamide

The procedure is as in Step C of Example 1, starting from the compound obtained in Example 13. The title compound is recrystallised from acetonitrile and obtained in the form of a white solid.

Melting Point: 175-1 76° C.

Elemental Microanalysis:

| % | C | H | N |
|---|---|---|---|
| Calculated: | 70.57 | 7.11 | 8.23 |
| Found: | 70.48 | 7.32 | 8.51 |

### Example 19

### N-[3-[Benzyl(methyl)amino]-2-(7-methoxy-1-naphthyl)propyl]-acetamide

The compound obtained in Example 8 (10 mmol) and N-methylbenzylamine (30 mmol) are dissolved in 60 ml of anhydrous tetrahydrofuran. The reaction mixture is placed under a current of argon and refluxed for 24 hours. When the reaction is complete, the mixture is concentrated in vacuo and then poured into water. The aqueous phase is extracted twice with 50 ml of ether, and the organic phase is washed with hydrochloric acid solution (2M). The aqueous phase is then rendered alkaline using sodium hydroxide solution (2M) and is then extracted twice with 50 ml of ether. The ether phase is washed with water, dried and evaporated under reduced pressure. The oil obtained is purified on silica gel using a mixture of acetone/cyclohexane (50/50) as eluant. The solid obtained after evaporation of the pure fractions is filtered off from a mixture of ether/petroleum ether under suction and is then recrystallised from diisopropyl ether.

Melting Point: 100-102° C.

### Example 20

### N-[2-(7-Methoxy-1-naphthyl)-3-(methylamino)propyl]acetamide

The compound obtained in Example 19 (2.6 mmol) is dissolved in 40 ml of methanol, and palladium-on-carbon (a spatula tip) is added to the solution. The reaction mixture is stirred under hydrogen at atmospheric pressure at ambient temperature for 24 hours. When the reaction is complete, the mixture is filtered and then concentrated under reduced pressure and poured into water. The aqueous phase is then extracted twice with 40 ml of ether, and the organic phase is washed with water, dried and then treated with ether saturated with HCl. The precipitate formed is filtered off from ether under suction and recrystallised from acetone to yield the title compound in the form of a white solid.

Melting Point: 126-128° C.

### Example 21

### N-{2-(7-Methoxy-1-naphthyl)-3-[(methylsulphonyl)amino]propyl}-propanamide

The procedure is as in Step C of Example 1, starting from the compound obtained in Example 14 and replacing the cyclopropanoyl chloride by mesyl chloride. The title compound is recrystallised from acetonitrile and obtained in the form of a white solid.

Melting Point: 140-142° C.

Elemental Microanalysis:

| % | C | H | N |
|---|---|---|---|
| Calculated: | 59.32 | 6.64 | 7.69 |
| Found: | 59.20 | 6.80 | 7.85 |

### Example 22

### N-Ethyl-N′-[3-hydroxy-2-(7-methoxy-1-naphthyl)propyl]urea

The compound obtained in Step B of Example 1 (5.6 mmol) is dissolved in 40 ml of tetrahydrofuran in the presence of triethylamine (1.56 mmol), and ethyl isocyanate (2.5 mmol) is added. The mixture is stirred at ambient temperature for 10 minutes, concentrated under reduced pressure and poured into water. The aqueous phase is extracted twice with 60 ml of ether; the organic phase is washed with hydrochloric acid solution (1M) and is then washed with water, dried and evaporated. The oil obtained is precipitated from a mixture of ether/petroleum ether (50/50); the precipitate formed is filtered off under suction and then recrystallised from acetonitrile to yield the title compound in the form of a white solid.

Melting Point: 120-122° C.

Elemental Microanalysis:

| % | C | H | N |
|---|---|---|---|
| Calculated: | 67.53 | 7.33 | 9.26 |
| Found: | 67.47 | 7.21 | 9.17 |

### Example 23

### N-[2-(3-Bromo-7-methoxy-1-naphthyl)-3-hydroxypropyl]acetamide

The procedure is as in Example 1, replacing the (7-methoxy-naphth-1-yl)acetonitrile in Step A by (3-bromo-7-methoxy-naphth-1-yl)acetonitrile, and the cyclopropanoyl chloride in Step C by acetyl chloride. The title compound is recrystallised from acetonitrile and obtained in the form of a white solid.

Melting Point: 129-131° C.

### Example 24

### N-[2-(3-Allyl-7-methoxy-1-naphthyl)-3-hydroxypropyl]acetamide

The procedure is as in Example 23, replacing the (7-methoxy-naphth-1-yl)acetonitrile in Step A by (3-allyl-7-methoxy-naphth-1-yl)acetonitrile. The title compound is recrystallised from acetonitrile and obtained in the form of a white solid.

Melting Point: 142-144° C.

### Example 25

### N-[2-(3-Allyl-7-methoxy-1-naphthyl)-3-hydroxypropyl]acetamide

The procedure is as in Example 23, replacing the (7-methoxy-naphth-1-yl)acetonitrile in Step A by (7-methoxy-3-vinyl-naphth-1-yl)acetonitrile. The title compound is recrystallised from acetonitrile and obtained in the form of a white solid.

Melting Point: 109-111° C.

### Example 26

### N-[4-Hydroxy-2-(7-methoxy-1-naphthyl)butyl]cyclopropane-carboxamide

### Step A: 4-Amino-3-(7-methoxy-1-naphthyl)-1-butanol hydrochloride

Aluminium chloride (80 mmol), dissolved in 200 ml of anhydrous ether, is added to a suspension of lithium aluminium hydride (80 mmol) at 0° C. in 300 ml of anhydrous ether. After stirring for 10 minutes, the compound obtained in Step B of Example 7 (20 mmol), dissolved in 200 ml of anhydrous ether, is added. After 30 minutes, the mixture is hydrolysed, in the cold state and with caution, using sodium hydroxide solution (250 mmol). The inorganic precipitate formed is then filtered off and washed with copious amounts of ether. The residue obtained after evaporation is taken up in water, and the aqueous phase is extracted with dichloromethane. The organic phase is then washed with water, dried and decoloured and is then treated with gaseous hydrogen chloride and evaporated. The oil obtained is precipitated from ethyl acetate, and the precipitate formed is filtered off under suction and then recrystallised to yield the title compound in the form of a white solid.

Melting Point: 164-166° C.

### Step B: N-[4-Hydroxy-2-(7-methoxy-1-naphthyl)butyl]cyclopropane-carboxamide

The compound obtained in Step A (20 mmol) is dissolved in a mixture of water/ethyl acetate (25 ml/75 ml) cooled to 0° C. Potassium carbonate (60 mmol) is added, and then cyclopropanecarboxylic acid chloride (26 mmol) is added dropwise to the reaction mixture. The batch is stirred vigorously at ambient temperature for 30 minutes. The two phases are separated and the organic phase is washed with 0.1M aqueous hydrochloric acid solution and then with water. After drying over magnesium sulphate, the organic phase is evaporated under reduced pressure. The residue obtained is recrystallised to yield the title compound in the form of a white solid.

Melting Point: 158-160° C.

### Example 27

### N-[4-Hydroxy-2-(7-methoxy-1-naphthyl)butyl]propanamide

The procedure is as in Example 26, replacing the cyclopropanecarboxylic acid chloride in Step B by propionic acid chloride. The title compound is obtained in the form of a white solid.

Melting Point: 123-125° C.

### Example 28

### 2-Fluoro-N-[4-hydroxy-2-(7-methoxy-1-naphthyl)butyl]acetamide

The procedure is as in Example 26, replacing the cyclopropanecarboxylic acid chloride in Step B by fluoroacetic acid chloride.

Melting Point: 96-98° C.

### Example 29

### N-[4-Hydroxy-2-(7-methoxy-1-naphthyl)butyl]cyclobutanecarboxamide

The procedure is as in Example 26, replacing the cyclopropanecarboxylic acid chloride in Step B by cyclobutanecarboxylic acid chloride. The title compound is obtained in the form of a white solid.

Melting Point: 113-115° C.

### Pharmacological Study

### EXAMPLE A

### Acute Toxicity Study

The acute toxicity was evaluated after oral administration to groups each comprising 8 mice (26±2 g). The animals were observed at regular intervals during the course of the first day, and daily for the two weeks following treatment. The LD₅₀ (dose that causes the death of 50% of the animals) was evaluated and demonstrated the low toxicity of the compounds of the invention.

### EXAMPLE B

### Forced Swimming Test

The compounds of the invention are tested in a behavioral model, the forced swimming test.

The apparatus is composed of a plexiglass cylinder filled with water. The animals are tested individually for a session of 6 minutes. At the start of each test, the animal is placed in the center of the cylinder. The time spent immobile is recorded. The animal is considered to be immobile when it stops struggling and remains immobile on the surface of the water, making only those movements which allow it to keep its head above water.

Following administration 40 minutes before the start of the test, the compounds of the invention significantly reduce the time spent immobile, which indicates their antidepressive activity.

### EXAMPLE C

### Melatonin MT₁ and MT₂ Receptor Binding Study

The MT₁ or MT₂ receptor binding experiments are carried out using 2-[¹²⁵I]-iodomelatonin as reference radioligand. The radioactivity retained is determined using a liquid scintillation counter.

Competitive binding experiments are then carried out in triplicate using the various test compounds. A range of different concentrations is tested for each compound. The results enable the binding affinities of the compounds tested (Kᵢ) to be determined.

The Kᵢ values found for the compounds of the invention accordingly demonstrate binding to one or other of the melatoninergic binding sites, those values being ≦10 μM.

As way of example, compound of Example 6 exhibits a Kᵢ (MT₁) of 4.9 nM and a Kᵢ (MT₂) of 8.9 nM.

### EXAMPLE D

### Serotoninergic 5-HT_{2C} Receptor Binding Study

The affinity of the compounds for the human 5-HT_{2C} receptor is evaluated on membrane preparations from CHO cells stably expressing that receptor.

Incubation is carried out in 50mM TRIS buffer, pH 7.4 containing 10 mM MgCl₂ and 0.1% BSA, in the presence of [³H]-mesulergine (InM) and 25 fmol/ml of receptor. Non-specific binding is determined in the presence of 10 μM mianserin.

The reaction is stopped by the addition of 50 mM TRIS buffer, pH 7.4 followed by a filtration step and 3 successive rinses: the radioactivity bound to the membranes remaining on the filters (GF/B pretreated with 0.1% PEI) is determined by liquid scintillation counting.

The results obtained show that the compounds of the invention have affinity for the 5-HT_{2C} receptor, with Kᵢ values <100μM.

As way of example, compound of Example 6 exhibits a Kᵢ (5-HT_{2C}) of 26 μM.

### EXAMPLE E

### Action of Compounds of the Invention On The Circadian Rhythms of Locomotive Activity of the Rat

The involvement of melatonin in influencing, by day/night alternation, the majority of physiological, biochemical and behavioral circadian rhythms has made it possible to establish a pharmacological model for use in the search for melatoninergic ligands.

The effects of the compounds are tested on numerous parameters and, in particular, on the circadian rhythms of locomotive activity, which constitute a reliable indicator of the activity of the endogenous circadian clock.

In this study, the effects of such compounds on a particular experimental model, namely the rat placed in temporal isolation (permanent darkness), is evaluated.

Experimental Protocol

One-month-old male rats are subjected, as soon as they arrive at the laboratory, to a light cycle of 12 hours' light per 24 hours (LD 12: 12).

After 2 to 3 weeks' adaptation, they are placed in cages fitted with a wheel connected to a recording system, in order to detect the phases of locomotive activity and thus monitor the nychthemeral rhythms (LD) or circadian rhythms (DD).

As soon as the rhythms recorded show a stable pattern during the light cycle LD 12: 12, the rats are placed in permanent darkness (DD).

Two to three weeks later, when the free course (rhythm reflecting that of the endogenous clock) is clearly established, the rats are given a daily administration of the compound to be tested.

The observations are made by means of visualisation of the rhythms of activity:
influence on the rhythms of activity by the light/dark cycle,
disappearance of the influence on the rhythms in permanent darkness,
influence on the activity by the daily administration of the compound; transitory or durable effect.

A Software Package Makes it Possible:
to measure the duration and intensity of the activity, the period of the rhythm of the animals during free course and during treatment,
possibly to demonstrate by spectral analysis the existence of circadian and non-circadian (for example ultradian) components.

Results

The compounds of the invention clearly appear to allow powerful action on the circadian rhythm via the melatoninergic system.

### EXAMPLE F

### Light/dark Cages Test

The compounds of the invention are tested in a behavioral model, the light/dark cages test, which allows the anxiolytic activity of the compounds to be demonstrated.

The apparatus consists of two polyvinyl boxes covered with plexiglass. One of the boxes is in darkness. A lamp is placed above the other box, yielding a light intensity of approximately 4000 lux in the center of the box. An opaque plastic tunnel separates the light box from the dark box. The animals are tested individually for a session of 5 minutes. The floor of each box is cleaned between each session. At the start of each test, the mouse is placed in the tunnel, facing the dark box. The time spent by the mouse in the illuminated box and the number of passages through the tunnel are recorded after the first entry into the dark box.

Following administration of the compounds 30 minutes before the start of the test, the compounds of the invention significantly increase the time spent in the illuminated cage and the number of passages through the tunnel, which demonstrates the anxiolytic activity of the compounds of the invention.

### EXAMPLE G

### Pharmaceutical Composition: Tablets

| | |
|---|---|
| 1000 tablets each containing 5 mg of N-[3-methoxy-2-(7- | 5 g |
| methoxy-1-naphthyl)-propyl]acetamide (Example 6) | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1. A compound selected from those of formula (I): 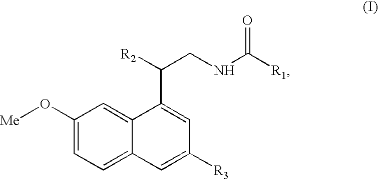
wherein:
R₁ represents a group R₄ or NHR₄, wherein R₄ represents a linear or branched (C₁-C₆)-alkyl group, a linear or branched (C₂-C₆)alkenyl group, a linear or branched (C₁-C₆)-haloalkyl group, a linear or branched (C₁-C₆)polyhaloalkyl group, a (C₃-C₈)cycloalkyl group, a (C₃-C₈)cycloalkyl-(C₁-C₆)alkyl group wherein the alkyl moiety may be linear or branched, an aryl group, an aryl-(C₁-C₆)alkyl group wherein the alkyl moiety may be linear or branched, a heteroaryl group or a heteroaryl-(C₁-C₆)alkyl group wherein the alkyl moiety may be linear or branched,
R₂ represents a linear or branched (C₁-C₆)alkyl group substituted by a linear or branched (C₁-C₆)alkoxy group, OH, OSO₂Me, N₃, NRR′, NHCOR″ or by NHSO₂R″, wherein R and R′, which may be the same or different, each represent a hydrogen atom or a linear or branched (C₁-C₆)alkyl group, a (C₃-C₈)cycloalkyl group, an aryl group or an aryl(C₁-C₆)alkyl group wherein the alkyl moiety may be linear or branched, or R and R′ together with the nitrogen atom carrying them form a 5- or 6-membered ring which may contain another hetero atom selected from nitrogen, oxygen and sulphur,
and R″ represents a linear or branched (C₁-C₆)alkyl group, a (C₃-C₈)cycloalkyl group, an aryl group or an aryl(C₁-C₆)alkyl group wherein the alkyl moiety may be linear or branched,
R₃ represents a hydrogen atom, a halogen atom, a linear or branched (C₁-C₆)alkyl group, or linear or branched (C₂-C₆)alkenyl group,
it being understood that:
when R₁ represents a methyl group and R₂ represents a hydroxymethyl group, then R₃ cannot represent a hydrogen atom,
an aryl group means a phenyl, naphthyl or biphenyl group,
a heteroaryl group means any aromatic mono- or bi-cyclic group containing from 1 to 3 hetero atoms selected from oxygen, sulphur and nitrogen,
wherein the aryl and heteroaryl groups may be unsubstituted or substituted by from 1 to 3 groups selected from linear or branched (C₁-C₆)alkyl, linear or branched (C₁-C₆)alkoxy, hydroxy, carboxy, formyl, nitro, cyano, linear or branched (C₁-C₆) haloalkyl, linear or branched (C₁-C₆)polyhaloalkyl, alkyloxycarbonyl and halogen atoms,
its enantiomers and diastereoisomers, and addition salts thereof with a pharmaceutically acceptable acid or base.

2. The compound of claim 1, wherein R₁ represents an alkyl group, its enantiomers and diastereoisomers, and addition salts thereof with a pharmaceutically acceptable acid or base.

3. The compound of claim 1, wherein R₂ represents an alkyl group substituted by a hydroxy group, its enantiomers and diastereoisomers, and addition salts thereof with a pharmaceutically acceptable acid or base.

4. The compound of claim 1, wherein R₂ represents an alkyl group substituted by an alkoxy group, its enantiomers and diastereoisomers, and addition salts thereof with a pharmaceutically acceptable acid or base.

5. The compound of claim 1, wherein R₃ represents a hydrogen atom, its enantiomers and diastereoisomers, and addition salts thereof with a pharmaceutically acceptable acid or base.

6. The compound of claim 1, which is selected from N-[3-methoxy-2-(7-methoxy-1-naphthyl)propyl]acetamide, its enantiomers and diastereoisomers, and addition salts thereof with a pharmaceutically acceptable acid or base.

7. The compound of claim 1, which is selected from N-[4-hydroxy-2-(7-methoxy-1-naphthyl)butyl]acetamide, its enantiomers and diastereoisomers, and addition salts thereof with a pharmaceutically acceptable acid or base.

8. The compound of claim 1, which is selected from N-[4-hydroxy-2-(7-methoxy-1-naphthyl)butyl]propanamide, its enantiomers and diastereoisomers, and addition salts thereof with a pharmaceutically acceptable acid or base.

9. A pharmaceutical composition comprising a compound of claim 1 or an addition salt thereof with a pharmaceutically acceptable acid or base alone or in combination with one or more pharmaceutically acceptable excipients.

10. A method for treating a living animal body, including a human, afflicted with a disorders of the melatoninergic system comprising the step of administering to the living animal body, including a human, an amount of the compound of claim 1 which is effective for treatment of the disorder.

11. The method of claim 10, wherein the disorder is selected from sleep disorders, stress, anxiety, major depression or seasonal affective disorder, cardiovascular pathologies, pathologies of the digestive system, insomnia and fatigue due to jetlag, schizophrenia, panic attacks, melancholia, appetite disorders, obesity, insomnia, psychotic disorders, epilepsy, diabetes, Parkinson's disease, senile dementia, various disorders associated with normal or pathological aging, migraine, memory loss, Alzheimer's disease, cerebral circulation disorders or sexual dysfunctions, as ovulation-inhibitors or immunomodulators, or for the treatment of cancers.
